(19) United States
(12) Patent Application Publication
(10) Pub. No.: US 2017/0194124 A1
(43) Pub. Date: Jul. 6, 2017

(54) X-RAY DELIVERY

(71) Applicant: VARIAN MEDICAL SYSTEMS, Palo Alto, CA (US)

(72) Inventors: Arundhuti Ganguly, San Jose, CA (US); Ivan Mollov, Mountain View, CA (US)

(21) Appl. No.: 14/989,313

(22) Filed: Jan. 6, 2016

(51) Int. Cl. H01J 35/06; A61N 5/10; H01J 35/14; G21K 1/02; H01J 35/08; H01J 35/18

(52) U.S. Cl. CPC H01J 35/06; H01J 35/08; H01J 35/18; H01J 35/14; G21K 1/02; A61N 5/10

(57) ABSTRACT

An X-ray assembly may include a housing, an anode, and a cathode assembly. The anode may be located at least partially within the housing. The anode may include a target area configured such that X-rays generated at the target area form an area-source X-ray beam. The cathode assembly may be located at least partially within the housing and may be positioned to deliver electrons to the target area of the anode from multiple directions relative to the target area, the multiple directions including at least two substantially opposite directions relative to the target area.

### BACKGROUND

Field

The embodiments discussed herein are related to X-ray generation and delivery.

Relevant Technology

An X-ray generating apparatus, or X-ray tube, conventionally includes a vacuum enclosure with an anode assembly and a cathode assembly spaced therebetween. The cathode assembly may include an electron emitting cathode, which is disposed so as to direct a beam of electrons onto a focal spot of an anode target of the anode assembly. In operation, electrons emitted by the cathode are accelerated towards the anode target by a high voltage applied between the cathode and the anode target. The accelerated electrons impinge on the focal spot area of the anode target with sufficient kinetic energy to generate a beam of x-rays which passes through a window in the vacuum enclosure.

X-ray tubes are used in a variety of industrial and medical applications. For example, X-ray tubes are employed in medical diagnostic examination, radiation therapy, semiconductor fabrication, and material analysis. In particular, radiation therapy for cancer treatment has been in use for decades. Conventional radiation therapy systems may employ an X-ray tube configured to generate a point-source X-ray beam, where X-rays may be emitted from the target in a generally conical pattern, which may be initially confined to a generally rectangular beam by a collimator having moveable, x-ray blocking “jaws” in the head of the system. Rarely, however, can the system jaws alone be used to implement a suitable treatment plan.

The subject matter claimed herein is not limited to embodiments that solve any disadvantages or that operate only in environments such as those described above. Rather, this background is only provided to illustrate one example technology area where some embodiments described herein may be practiced.

### SUMMARY

Embodiments may generally relate to X-ray generation and delivery. In particular, embodiments may relate to systems, devices, and/or methods for X-ray generation and delivery.

This Summary introduces a selection of concepts in a simplified form that are further described below in the Detailed Description. This Summary is not intended to identify key features or essential characteristics of the claimed subject matter, nor is it intended to be used as an aid in determining the scope of the claimed subject matter.

In some embodiments, an X-ray assembly may include a housing, an anode, and a cathode assembly. The anode may be located at least partially within the housing. The anode may include a target area configured such that X-rays generated at the target area form an area-source X-ray beam. The cathode assembly may be located at least partially within the housing and may be positioned to deliver electrons to the target area of the anode from multiple directions relative to the target area, the multiple directions including at least two substantially opposite directions relative to the target area.

In some embodiments, a system may include an X-ray assembly and a focusing collimator. The X-ray assembly may include a housing, an anode, and a cathode. The anode may be located at least partially within the housing. The anode may include a target area. The cathode assembly may be located at least partially within the housing. The cathode assembly may include a cathode and a focusing electrode. The cathode assembly may be positioned to deliver electrons to the target area of the anode from multiple directions relative to the target area. The focusing collimator may include at least one substantially frustoconical passage.

Additional features and advantages will be set forth in the description which follows, and in part will be obvious from the description, or may be learned by the practice of the embodiments. The features and advantages of the embodiments will be realized and obtained by means of the instruments and combinations particularly pointed out in the claims. These and other features will become more fully apparent from the following description and claims, or may be learned by the practice of the embodiments as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a cross-sectional side view of an example point-source X-ray system;

FIG. 2A illustrates a cross-sectional side view of an example area-source X-ray system;

FIG. 2B illustrates a bottom view of an example target area and cathode assembly of the X-ray system of FIG. 2A;

FIG. 3 illustrates another example cathode assembly that may be employed in the X-ray system of FIG. 2A;

FIG. 4A illustrates a cross-sectional side view of another example area-source X-ray system;

FIG. 4B illustrates a bottom view of an example target area and cathode assembly of the X-ray system of FIG. 4A;

FIG. 5 illustrates a cross-sectional side view of another example area-source X-ray system;

FIG. 6 illustrates a cross-sectional side view of another example area-source X-ray system;

FIG. 7 illustrates a cross-sectional side view of another example area-source X-ray and collimator system;

FIG. 8 illustrates a cross-sectional side view of another example area-source X-ray and collimator system;

FIG. 9 illustrates a cross-sectional side view of another example area-source X-ray and collimator system;

FIG. 10 illustrates a cross-sectional side view of another example area-source X-ray and collimator system;

FIG. 11 illustrates a representation of another example area-source X-ray and collimator system;

FIG. 12 is a diagram of an example X-ray system; and

FIG. 13 is a diagram of another example X-ray system.

### DESCRIPTION OF EMBODIMENTS

Reference will now be made to the figures wherein like structures will be provided with like reference designations. The drawings are diagrammatic and schematic representations and, accordingly, are not limiting of the scope of the claimed subject matter, nor are the drawings necessarily drawn to scale.

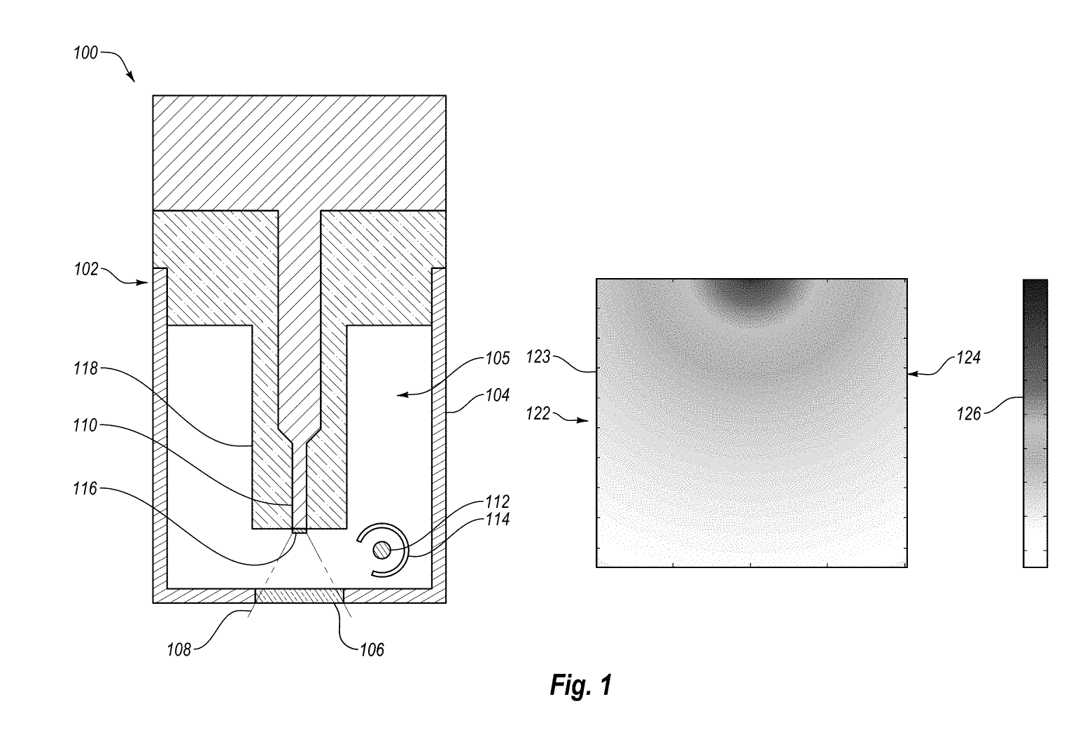

FIG. 1 illustrates a cross-sectional side view of a conventional point-source X-ray system 100. The system 100 includes an X-ray assembly 102. The X-ray assembly 102 includes a housing 104, which may enclose a vacuum enclosure 105 and which may include an X-ray window 106. The X-ray assembly 102 includes an anode 110 and a single cathode 112.

The cathode 112 may emit electrons, which may impinge the anode 110 at a target area 116. An isolator 118 may discourage electrons from impinging the anode 110 at a location other than the target area 116. The X-ray assembly 102 may include a focusing electrode 114 for steering, focusing, or otherwise influencing the paths of electrons emitted by the cathode 112. The electrons emitted by the cathode 112 may impinge the target area 116 of the anode 110 and may generally produce an X-ray beam 108 that may pass through the X-ray window 106. In some configurations, the X-ray window 106 may be a portion of the housing 104 that the X-ray beam 108 passes through. In some other configurations, the X-ray window 106 may be different from the housing 104 in some way, such as being made from a different material than the housing 104 or the like.

The target area 116 of the anode 110 may have a surface area of less than a few square millimeters (mm). For example, in some configurations, the target area 116 of the anode 110 may have a surface area of about 1 mm. Thus, for example, the X-ray beam 108 may appear to be originating from a relatively small area. Thus, for practical purposes, the X-ray beam 108 may behave as if originating from a point source.

A radiation intensity diagram 122 illustrates a cross-section of a relative intensity 124 of the radiation generated at the target area 116. The relative intensity 124 is shown according to a color scale 126.

As demonstrated in the radiation intensity diagram 122, levels of radiation intensity 123 generated by the point-source X-ray system 100 may be relatively weak. Furthermore, the sections of relatively consistent intensity may be approximately small and may be relatively shaped as a spherical cap. Put another way, the spherical radiation intensity may not include an area with a relatively consistent, flat intensity.

Thus, for example, the X-ray system 100 may produce a relatively low-power X-ray beam 108. Point-source X-ray systems may be used for radiation treatment of surfaces and/or internal volumes of a body. However, using the relatively low-power X-ray beam for radiation therapy may lead to longer exposure times and/or relatively high exposure to areas of the body that do not require treatment. As a result, treatment via point-source X-ray systems may be accompanied by a risk of damaging healthy portions of a body in the course of treating unhealthy portions.

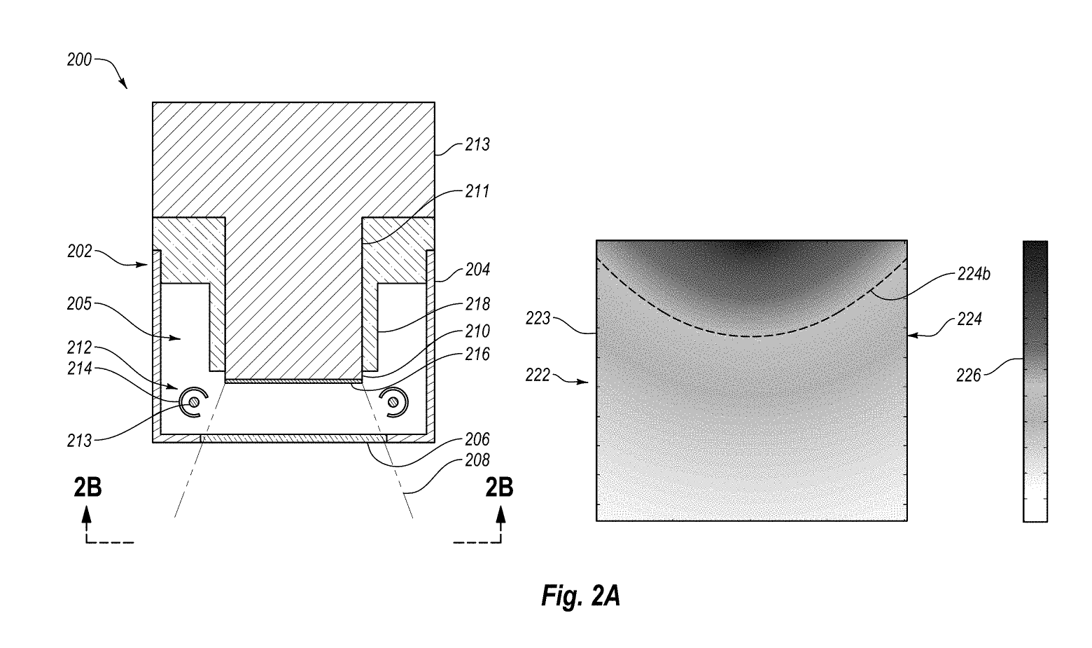

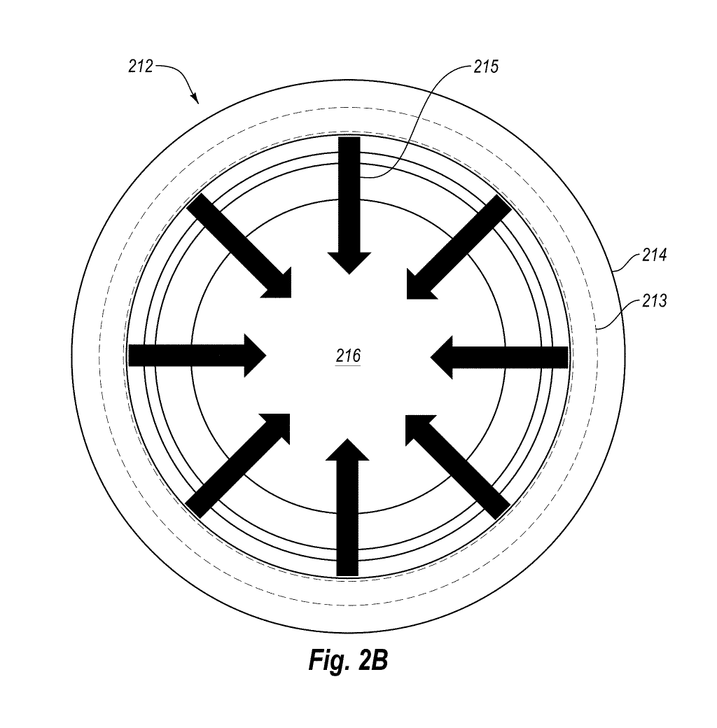

FIG. 2A illustrates a cross-sectional side view of an area-source X-ray system 200. The system 200 includes an X-ray assembly 202. The assembly 202 may include a housing 204, a vacuum enclosure 205, an X-ray window 206, and/or an isolator 218 generally corresponding to the housing 104, the vacuum enclosure 105, the X-ray window 106, and/or the isolator 118, respectively, of FIG. 1. The assembly 202 includes an anode 210 having a target area 216. The target area 216 of the anode 210 may have a relatively large surface area. By way of example, the surface area of the target area 216 may be greater than 10 square mm. In some embodiments, the surface area of the target area 216 may be greater than 50 square mm. For example, in some configurations, the target area 216 may include a disk shape having a diameter of 10 mm, 20 mm, or some other length.

The assembly 202 includes a cathode assembly 212. The cathode assembly 212 includes a cathode 213 and may further include a focusing electrode 214. The cathode 213 and/or the focusing electrode 214 may be positioned to deliver electrons to the target area 216 of the anode 210 from multiple directions relative to the anode 210. For example, the cathode assembly 212 may be positioned on opposite sides of the target area of the anode 210. In some embodiments, the cathode 213 and/or the focusing electrode 214 may be positioned at an offset relative to an edge of the target area 216. For example, the cathode assembly 212 may include a substantially circular shape positioned around the target area 216 having a dish shape. In some embodiments, by delivering electrons to the target area 216 from multiple directions, the target area 216 may be impinged by electrons over its relatively large area. Thus, for example, an X-ray beam 208 may be produced over the relatively large area of the target area 216. In this and other embodiments, the cathode assembly 212 may be omitted and the target area 216 may be replaced or covered with a radioactive isotope. Thus, for example, X-ray generation may occur without the cathode assembly 212 or an electron stream.

A radiation intensity diagram 222 illustrates a cross-section of a relative radiation intensity 224 of X-rays generated at the target area 216. The relative intensity 224 is shown according to a color scale 226.

As illustrated in the radiation intensity diagram 222, the highest relative level of radiation intensity 223 generated by the X-ray system 200 may be relatively higher through a relatively larger volume than conventional point-source X-ray systems, such as the X-ray system 100 of FIG. 1. Furthermore, the X-ray system 200 may generate sections of relatively equivalent intensity, which may be relatively larger and/or flatter than conventional point-source X-ray systems, such as the X-ray system 100 of FIG. 1. For example, the radiation intensity at the location represented by the line 224b may be relatively equivalent.

An area X-ray assembly, such as the X-ray system 200 of FIG. 2A may be used in medical treatments. For example, the X-ray assemblies may produce X-rays for treating cancerous tissue or the like. In this and other embodiments, an area X-ray assembly may be used for treating a surface of a body, such as an area of skin of a human patient. Alternately or additionally, an area X-ray assembly may be used for sterilizing food, blood samples, medical instruments, or the like.

FIG. 2B illustrates a bottom view of the target area 216 and the cathode assembly 212 of the X-ray system 200 of FIG. 2A. In some embodiments, the cathode assembly 212 may be positioned at an offset relative to an edge of the target area 216. The cathode assembly 212 may include a single cathode 213 and/or a single focusing electrode 214. The cathode assembly 212 may deliver an electron stream, represented by arrows 215, to the target area 216 from multiple directions relative to the target area 216. Although represented by the discrete arrows 215, the cathode assembly 212 may deliver the electron stream from along the length of the cathode assembly 212. In some embodiments, the cathode 213 and the focusing electrode 214 may be configured to direct the electron stream relatively consistently over the target area 216.

In some embodiments, the cathode 213 and the focusing electrode 214 may be configured to vary the intensity of the electron stream over the target area 216. Varying the intensity of the electron stream over the target area 216 may, in turn, vary the intensity of the X-rays generated across the target area 216.

The target area 216 may include a disk shape, as illustrated. In some embodiments, the target area 216 may include any other suitable shape, such as another elliptical shape, a planar donut shape, a rectangular or other polygon shape, or the like or any combination thereof. Furthermore, in this and other embodiments, the target area 216 may include a three-dimensional shape in order to shape the X-ray beam intensity profile. The cathode assembly 212 may be shaped to correspond to the shape of the target area 216 and/or to deliver electrons to the target area 216 in a desired manner. Furthermore, in this and other embodiments, the anode 210 may include multiple point-source target areas arranged to perform collectively in a manner similar to an area-source target area.

In this and other embodiments, the shapes of the target area and the anode are provided as an example. The shape of the target area may generally include any shape suitable for producing an X-ray beam such that the X-ray beam originates from an area source. In this and other embodiments, the configuration and position of the cathode assembly are provided as an example. The cathode assembly may generally be configured and positioned to deliver electrons over the target area. In some embodiments, the cathode assembly may be configured and positioned to deliver electrons to the target area from multiple directions relative to the target area, including a continuous cathode around the target area of the anode. In this and other embodiments, the multiple directions may include substantially opposite directions relative to the target area. For example, portions of the cathode assembly located on opposite sides of the target area may deliver electrons to the target area from substantially opposite directions relative to the target area.

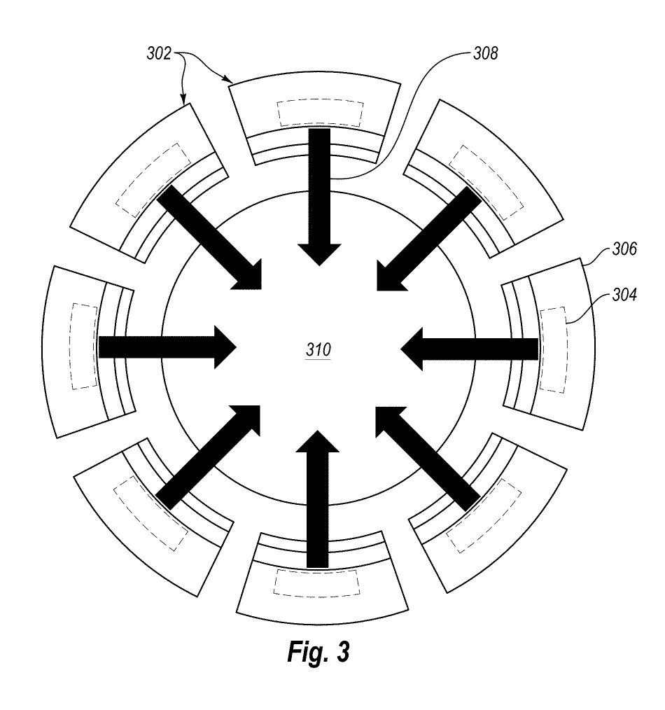

FIG. 3 illustrates another cathode assembly 302 that may be employed in the X-ray system 200 of FIG. 2A. In some embodiments, the cathode assembly 302 may include multiple cathodes 304. Alternately or additionally, the cathode assembly 302 may include multiple focusing electrodes 306. Although the cathode assembly 302 is illustrated as including 8 cathodes 304 and 8 focusing electrodes 306, the cathode assembly 302 may include more cathodes 304 and/or focusing electrodes 306 or fewer cathodes 304 and/or focusing electrodes 306.

The cathodes 304 and/or the focusing electrodes 306 may deliver multiple electron streams, represented by the arrows 308, to a target area 310 from multiple directions relative to the target area 310. In some embodiments, the cathodes 304 and/or the focusing electrodes 306 may be configured to direct the electron streams relatively consistently over the target area 310. The cathodes 304 and the focusing electrodes 306 may be positioned to correspond to the shape of the target area 310 and/or to deliver electrons to the target area 310 in a desired manner.

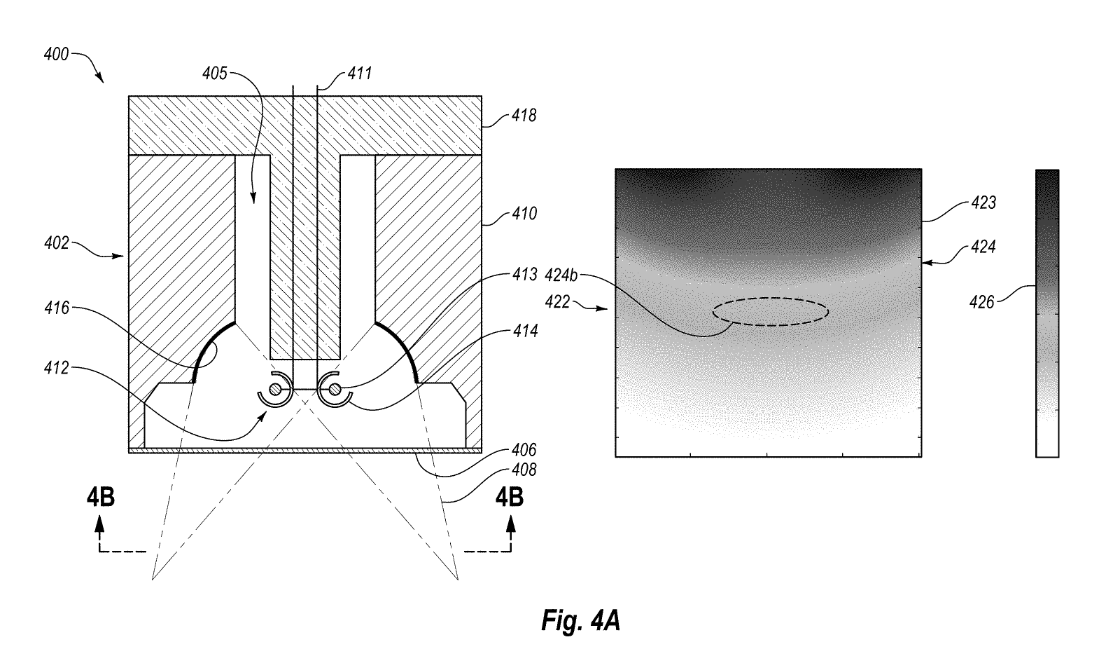

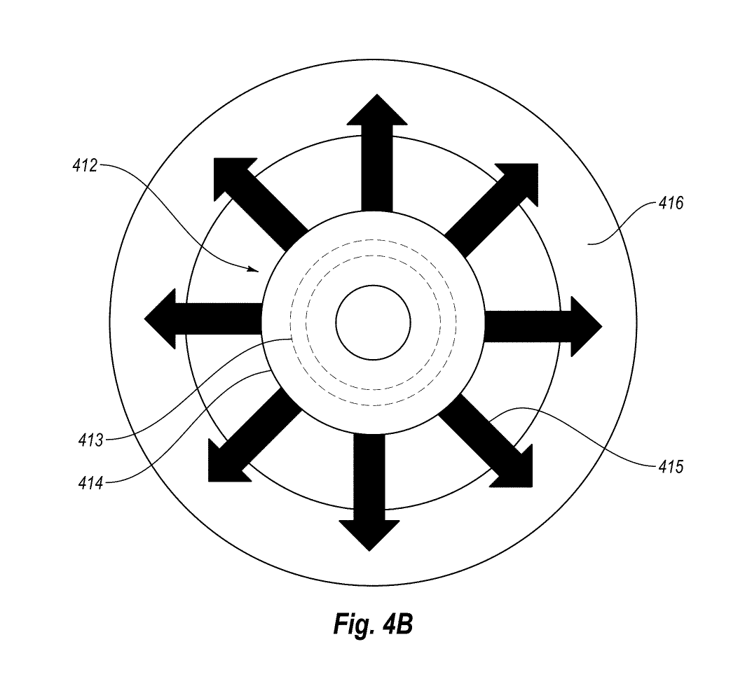

FIG. 4A illustrates a cross-sectional side view of another area-source X-ray system 400. The system 400 includes an X-ray assembly 402. The assembly 402 may include a vacuum enclosure 405, an X-ray window 406, and/or an isolator 418.

The assembly 402 includes an anode 410 having a target area 416. The target area 416 may include a concave surface oriented in a ring shape. The target area 416 may be described herein as a concave ring shape or a non-planar donut shape. The assembly 402 also includes a cathode assembly 412. The cathode assembly 412 includes a cathode 413 and may include a focusing electrode 414. Conductive lines 411 may be used for power and control of the cathode assembly 412. In some embodiments, the target area 416, the anode 410 and/or the cathode 413 and focusing electrode 414 may be circular about a vertical axis. As illustrated in FIG. 4A, the anode 410 encircles the cathode assembly 412. The assembly 402 may produce an X-ray beam 408.

A radiation intensity diagram 422 illustrates a relative intensity 424 of X-rays generated by the X-ray system 400. The relative intensity 424 is shown according to a color scale 426.

As illustrated in the radiation intensity diagram 422, the highest relative level of radiation intensity 423 generated by the X-ray system 400 may be relatively higher through a relatively larger volume than conventional point-source X-ray systems, such as the X-ray system 100 of FIG. 1. Furthermore, the X-ray system 400 may generate sections of relatively equivalent intensity, which may be relatively larger, flatter, and/or relatively more closely located than conventional point-source X-ray systems, such as the X-ray system 100 of FIG. 1. For example, the radiation intensity at the location enclosed by the line 424b may be subject to relatively predictable and/or controllable radiation intensity. Thus, for example, the radiation intensity at locations of relatively predicable and/or controllable radiation intensity may be advantageously employed for medical treatments and/or other radiation treatments.

FIG. 4B illustrates a bottom view of the target area 416 and the cathode assembly 412 of the X-ray system 400 of FIG. 4A. In some embodiments, the cathode assembly 412 may be positioned at an offset relative to an edge of the target area 416. For example, the cathode assembly 412 may be positioned at an offset relative to an interior edge of the target area 416. As illustrated in FIG. 4B, the cathode assembly 412 may include a single cathode 413 and/or a single focusing electrode 414, analogous to the cathode assembly 212 of FIG. 2B. The cathode assembly 412 may deliver an electron stream, represented by arrow 415 to the target area 416 from multiple directions relative to the target area 416. In this and other embodiments, the cathode assembly 412 may alternatively include multiple cathodes and/or multiple focusing electrodes analogous to the cathode assembly 302 of FIG. 3.

Referring collectively to FIGS. 4A and 4B, the target area 416 may include a concave surface oriented in a ring shape, as illustrated. In some embodiments, the target area 416 may include any other suitable shape, such as other surface shapes, other elliptical strip shapes, or the like or any combination thereof. Alternatively, the target area 416 may include other shapes in which a cathode assembly 412 may be positioned within an interior edge of the target area 416.

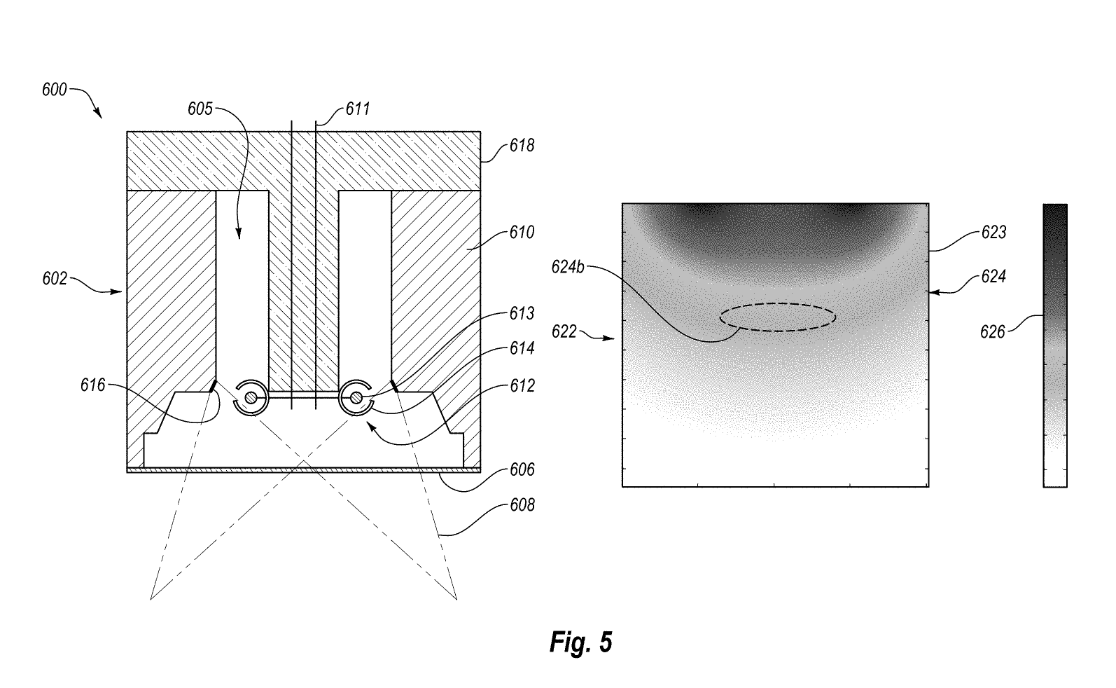

FIG. 5 illustrates a cross-sectional side view of another example area-source X-ray system 600 including an X-ray assembly 602. The assembly 602 may include a vacuum enclosure 605, an X-ray window 606, conductive lines 611, a cathode assembly 612, a cathode 613, a focusing electrode 614, and/or an isolator 618 generally corresponding to the vacuum enclosure 405, the X-ray window 406, the conductive lines 411, the cathode assembly 412, the cathode 413, the focusing electrode 414, and/or the isolator 418 of FIG. 4A. The assembly 602 includes an anode 610 having a target area 616. The target area 616 may include a relatively narrow strip frustoconical in shape. The target area 616 shape may be described herein as a non-planar ring shape. Alternately, the target area 616 may include other suitable shapes, such as a planar ring shape. In some embodiments, the target area 616 may be relatively narrow such that an emitted X-ray beam may be selectively attenuated by a collimator with relative efficiency. By way of example, collimators such as those described herein may be used to selectively attenuate an X-ray beam emitted from a relatively narrow target area relatively efficiently. In some embodiments, the anode 610 may include multiple target areas having a narrow ring shape analogous to the target area 616. The assembly 602 may produce an X-ray beam 608.

A radiation intensity diagram 622 illustrates a relative intensity 624 of X-rays generated at the target area 616. The relative intensity 624 is shown according to a color scale 626.

As illustrated in the radiation intensity diagram 622, the highest relative level of radiation intensity 623 generated by the X-ray system 600 may be relatively higher through a relatively larger volume than conventional point-source X-ray systems, such as the X-ray system 100 of FIG. 1. Furthermore, the X-ray system 600 may generate sections of relatively equivalent intensity, which may be relatively larger, flatter, and/or relatively more closely located than conventional point-source X-ray systems, such as the X-ray system 100 of FIG. 1. For example, the radiation intensity at the location enclosed by the line 624b may be subject to relatively predictable and/or controllable radiation intensity. Thus, for example, the radiation intensity at locations of relatively predictable and/or controllable radiation intensity may be advantageously employed for medical treatments and/or other radiation treatments.

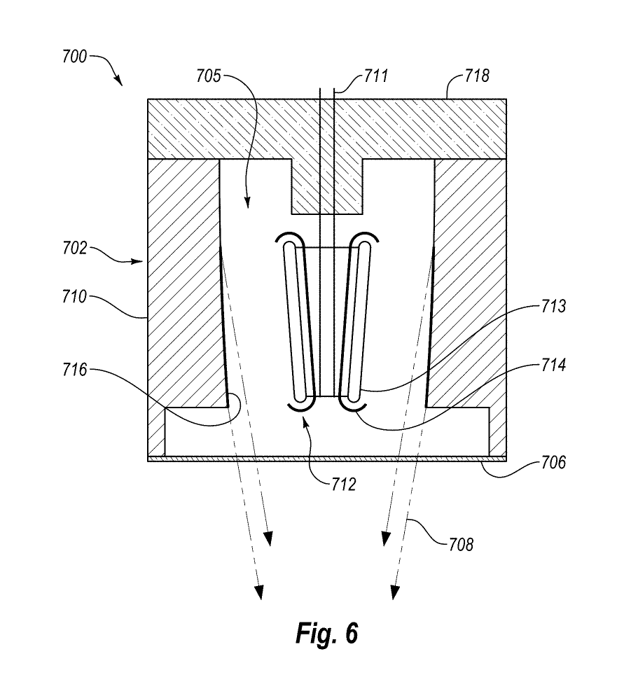

FIG. 6 illustrates a cross-sectional side view of another example area-source X-ray system 700 including an X-ray assembly 702. The assembly 702 may include a vacuum enclosure 705, an X-ray window 706, conductive lines 711, and/or an isolator 718 generally corresponding to the vacuum enclosure 405, the X-ray window 406, the conductive lines 411, and/or the isolator 418 of FIG. 4A. The assembly 702 includes an anode 710 having a target area 716. The target area 716 may be frustoconical or cylindrical in shape. The target area 716 shape may be described herein as a broad ring shape. Alternately, the target area 716 may include other suitable shapes. As illustrated, the target area 716 may include a smaller end and a larger end, with the smaller end positioned relatively closer to the X-ray window 706 than the larger end. Alternately, the larger end may be positioned relatively closer to the X-ray window 706 than the smaller end.

The assembly 702 may include a cathode assembly 712. The cathode assembly 712 includes a cathode 713 and may further include a focusing electrode 714. The cathode assembly 712 may include a single cathode or multiple cathodes. The assembly 702 may produce an X-ray beam 708.

In some embodiments, the shape of a target area and/or the number of target areas of an anode of an X-ray system may correspond to a collimator to be employed with the X-ray assembly. For example, a target area corresponding to the target area 416 of FIG. 4A, the target area 616 of FIG. 5, the target area 716 of FIG. 6, or another target area shape may be used in an X-ray assembly for use with one or more collimators.

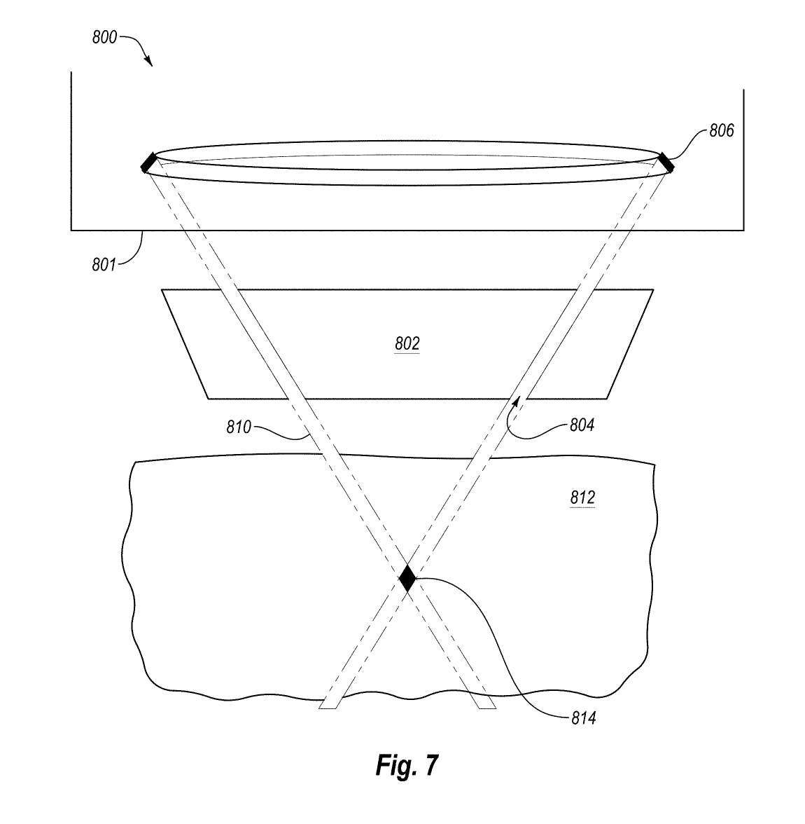

FIG. 7 illustrates a cross-sectional side view of another example area-source X-ray system 800. The system includes a collimator 802 having an X-ray passage 804. In this and other embodiments, portions of the collimator 802 other than the X-ray passage 804 may attenuate X-rays. The collimator 802 may be circular. Thus, for example, as illustrated, the X-ray passage 804 may have a frustoconical shape. In this and other embodiments, the X-ray passage 804 may include an opening in the collimator 802 with supports such as pins positioned to hold the sections of the collimator 802 in place relative to each other. Alternately or additionally, the X-ray passage 804 may include materials relatively transparent to X-rays, such as appropriate polymers or the like.

The collimator 802 may be positioned between an area-source X-ray assembly and a body 812. The area-source X-ray assembly may include, for example, a housing 801 and an anode having a target area 806. The target area 806 may generally correspond to the target area 616 of FIG. 5 or 716 of FIG. 6. The target area 806 is illustrated to demonstrate example three-dimensional attributes of the target area 806. Furthermore, the remaining X-ray assembly associated with the target area 806 is omitted for clarity. Alternately, the area-source X-ray assembly may include an anode having a differently shaped target area. Alternately or additionally, the collimator 802 may be used with one or more point-source X-ray assemblies and/or an anode including multiple point-source target area surfaces.

The target area 806 may produce an X-ray beam 810. Portions of the X-ray beam 810 that would be attenuated by the collimator 802 are omitted for clarity. The shape of the X-ray passage 804 may create a relatively cone-shaped X-ray beam 810 below the collimator 802 that meets at a treatment volume 814. In this and other embodiments, collimator 802 profile may be custom shaped to create a treatment volume 814 corresponding to a particular patient's particular tumor shape. Additionally, multiple collimators corresponding to the collimator 802 may be designed to create the desired treatment volume 814 shape from various perspectives relative to a patient's body. Thus, for example, multiple X-ray sources may be used with the multiple collimators to deliver relatively high radiation intensity to a particular treatment volume from multiple locations relative to a patient's body. In this and other embodiments, the collimator 802 may be manufactured via 3D printing processes and/or other rapid manufacturing processes.

Thus, for example, the collimator 802 may allow radiation treatment of a treatment volume 814 such as a tumor or the like, below a surface of the body 812 while limiting the radiation experienced by other portions of the body 812. For example, the treatment volume 814 may be exposed to higher radiation intensity than other parts of the body 812, which may allow treatment inside of the body 812 without damaging surrounding tissue. Furthermore, radiation treatment of the treatment volume 814 may be accomplished with a single X-ray assembly and collimator 802, which may be held stationary relative to the body 812.

In this and other embodiments, the X-ray system 800 may be used to perform radiation treatment by changing the relative position of the X-ray system 800 relative to a body, such as the body of a human patient, to be treated. In some embodiments, the X-ray system 800 may be moved dynamically (e.g., moved as the X-ray beam 810 is being produced) relative to the body. Alternately, the X-ray system 800 may be moved in a point-and-shoot manner (e.g., moved when the production of the X-ray beam 810 is ceased) relative to the body.

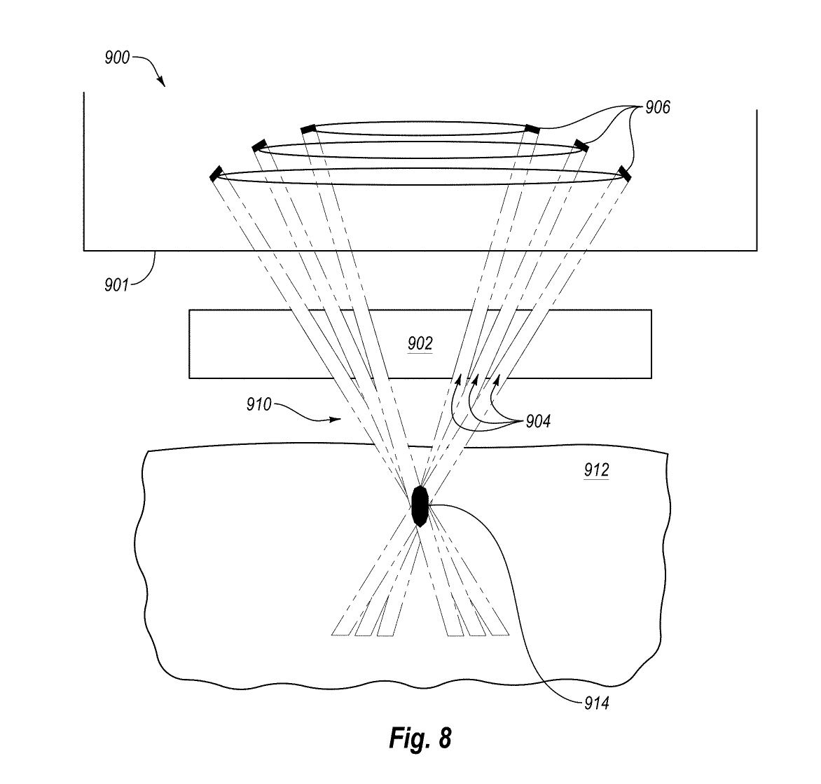

FIG. 8 illustrates a cross-sectional side view of another example area-source X-ray system 900. The system includes multiple X-ray passages 904. The collimator 902 may be circular. Thus, for example, as illustrated, each of the X-ray passages 904 may have a frustoconical shape.

The collimator 902 may be positioned between an area-source X-ray assembly and a body 912. The area-source X-ray assembly may include, for example, a housing 901 and an anode having multiple ring-shaped target areas 906. Alternately, the area-source X-ray assembly may include an anode having a differently shaped target area. The target areas 906 are illustrated to demonstrate example three-dimensional attributes of the target areas 906. Furthermore, the remaining X-ray assembly associated with the target areas 906 is omitted for clarity. The target areas 906 may produce X-ray beams 910. Portions of the X-ray beams 910 that would be attenuated by the collimator 902 are omitted for clarity.

The shape of each of the X-ray passages 904 may create a corresponding, relatively cone-shaped X-ray beam 910 below the collimator 902. The X-ray passages 904 are convergent. For example, each of the X-ray passages 904 may be positioned such that each of the relatively cone-shaped X-ray beams 910 meet approximately at substantially the same treatment volume 914 in a body 912.

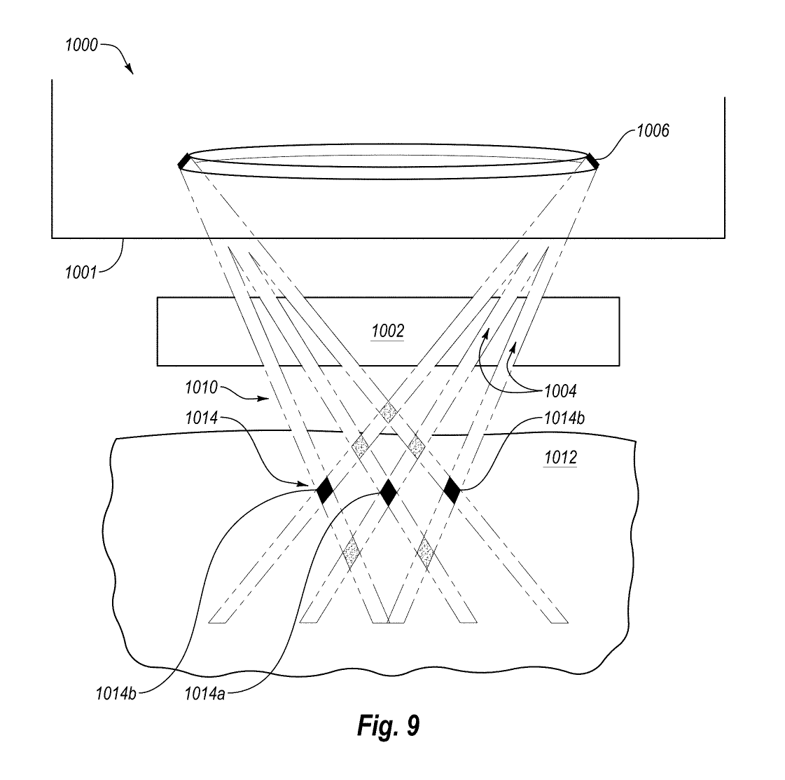

FIG. 9 illustrates a cross-sectional side view of another example area-source X-ray system 1000. The system includes multiple X-ray passages 1004. The collimator 1002 may be circular. Thus, for example, as illustrated, each of the X-ray passages 1004 may have a frustoconical shape. Alternatively, the X-ray passages 1004 may include other shapes to produce different treatment volumes 1014. In this and other embodiments, the collimator 1002 may include more or fewer X-ray passages 1004 to produce more or fewer treatment volumes 1014.

The collimator 1002 may be positioned between an area-source X-ray assembly and a body 1012. The area-source X-ray assembly may include, for example, a housing 1001 and an anode having a target area 1006. The target area 1006 may generally correspond to the target area 616 of FIG. 5. Alternately, the area-source X-ray assembly may include an anode having a differently shaped target area. The target area 1006 is illustrated to demonstrate example three-dimensional attributes of the target area 1006. Furthermore, the remaining X-ray assembly associated with the target areas 1006 is omitted for clarity. The target area 1006 may produce an X-ray beam 1010. Portions of the X-ray beams 1010 that would be attenuated by the collimator 1002 are omitted for clarity.

The shape of each of the X-ray passages 1004 may create a corresponding, relatively cone-shaped X-ray beam 1010 below the collimator 1002. The X-ray passages 1004 are divergent. For example, each of the X-ray passages 1004 may be positioned such that each of the relatively cone-shaped X-ray beams 1010 meet at multiple treatment volumes 1014 in a body 1012. In embodiments including relatively circular X-ray passages 1004, the treatment volumes 1014 may include a three-dimensional shape resembling a target volume 1014a encircled by a ring-shaped target volume 1014b. In some embodiments, the X-ray passages 1004 may be configured to create treatment volumes 1014 more closely or more distantly positioned.

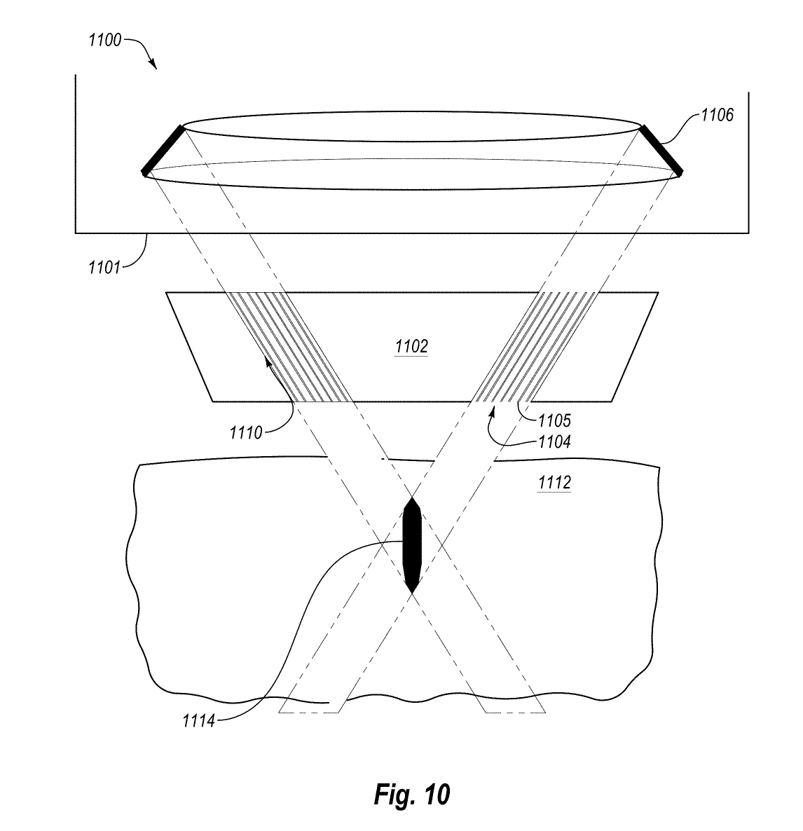

FIG. 10 illustrates a cross-sectional side view of another example area-source X-ray system 1100. The system includes multiple X-ray passages 1104. The collimator 1102 may be circular. Thus, for example, as illustrated, each of the X-ray passages 1104 may have a frustoconical shape.

The collimator 1102 may be positioned between an area-source X-ray assembly and a body 1112. The area-source X-ray assembly may include, for example, a housing 1101 and an anode having a target area 1106. The target area 1106 may generally correspond to the target area 416 of FIG. 4A or target area 716 of FIG. 6. Alternately, the area-source X-ray assembly may include an anode having a differently shaped target area. The target area 1106 is illustrated to demonstrate example three-dimensional attributes of the target area 1106. Furthermore, the remaining X-ray assembly associated with the target area 1106 is omitted for clarity. The target area 1106 may produce an X-ray beam 1110. Portions of the X-ray beam 1110 that would be attenuated by the collimator 1102 are omitted for clarity.

The shape of each of the X-ray passages 1104 may create a corresponding, relatively cone-shaped X-ray beam 1110 below the collimator 1102. The X-ray passages 1104 are parallel. Conceptually, the X-ray passages 1104 may alternately be considered to include a single, relatively large X-ray passage 1104 including multiple concentric walls 1105. By way of example, the parallel X-ray passages 1104 may result in a relatively laminar, relatively cone-shaped X-ray beam 1110 that meets at a relatively large treatment volume 1114 in a body 1112.

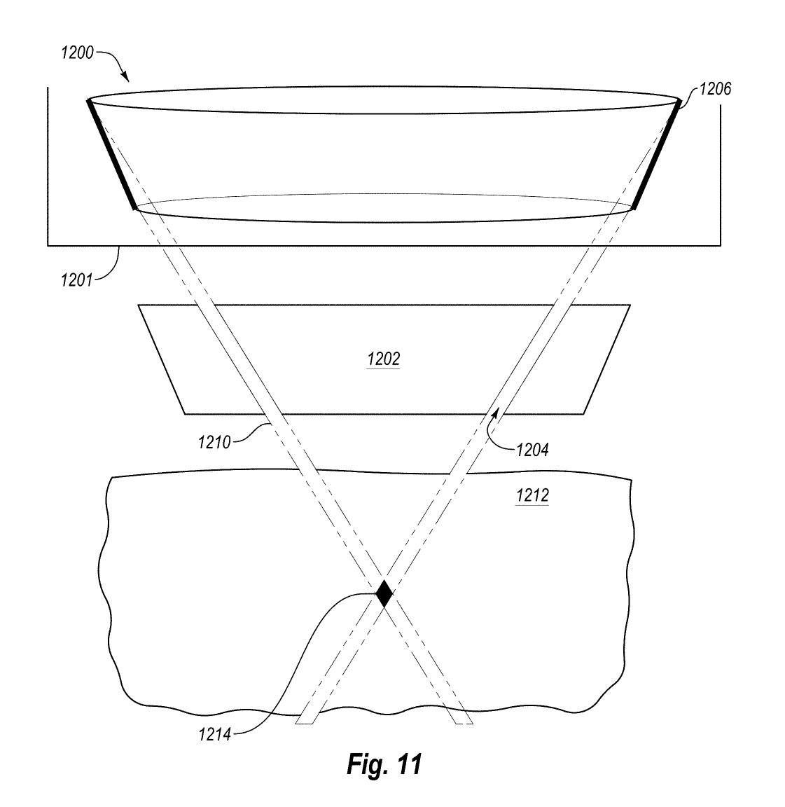

FIG. 11 illustrates a cross-sectional side view of another example area-source X-ray system 1200. The system includes an X-ray passage 1204. The collimator 1202 may be circular. Thus, for example, as illustrated, the X-ray passage 1204 may have a frustoconical shape.

The collimator 1202 may be positioned between an area-source X-ray assembly and a body 1212. The area-source X-ray assembly may include, for example, a housing 1201 and an anode having a target area 1206. The target area 1206 may generally correspond to the target area 716 of FIG. 6. Alternately, the area-source X-ray assembly may include an anode having a differently shaped target area. The target area 1206 is illustrated to demonstrate example three-dimensional attributes of the target area 1206. Furthermore, the remaining X-ray assembly associated with the target areas 1206 is omitted for clarity. The target area 1206 may produce an X-ray beam 1210. Portions of the X-ray beam 1210 that would be attenuated by the collimator 1202 are omitted for clarity.

The shape of the X-ray passage 1204 may create a corresponding, relatively cone-shaped X-ray beam 1210 below the collimator 1202. Furthermore, the frustoconical target area 1206 may produce a relatively frustoconical X-ray beam 1210. Thus, for example, a relatively high portion of the X-rays produced at the target area 1206 may be directed to the body 1212 and to the treatment volume 1214.

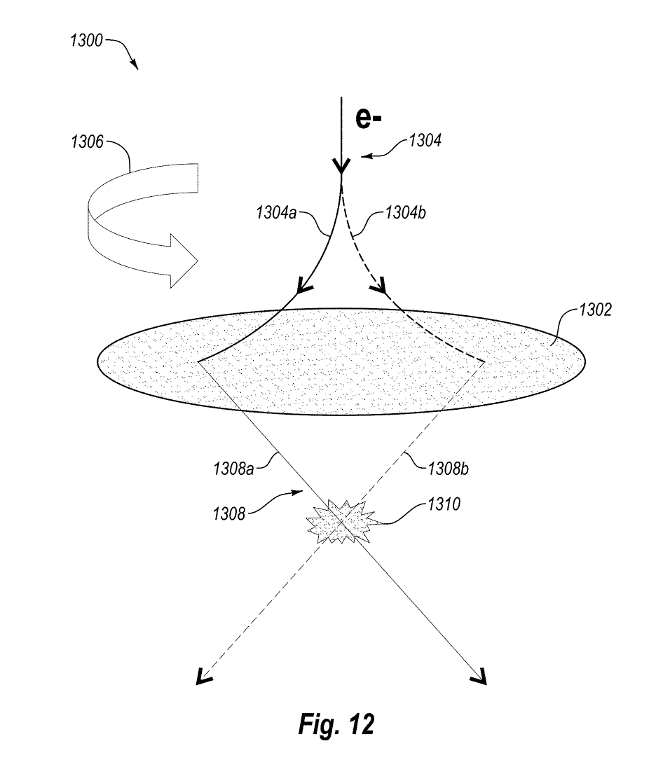

FIG. 12 is a diagram of an example X-ray system 1300. In some embodiments, the X-ray system 1300 may be used to create a treatment volume 1310. Optionally, the X-ray system 1300 may be used with a collimator, such as a collimator as described with reference to any of FIGS. 8-12.

The X-ray system 1300 may include a target area 1302 of an anode of an X-ray assembly. Portions of the X-ray assembly other than the target area 1302 and the collimator are omitted for clarity. The target area 1302 may be relatively thin, such that an electron beam 1304 striking one side of the target area 1302 may produce an X-ray beam 1308 on the other side of the target area 1302. The electron beam 1304 may be steered in a relatively circular motion, represented by arrow 1306. Thus, for example, the electron beam 1304 may strike the target area 1302 in a pattern approximating a circle and the X-ray beam 1308 may be produced in a pattern also approximating the circle. Each of the positions of X-ray beam 1308 may intersect at an approximate treatment volume 1310.

By way of example, at a first time, the electron beam 1304a may result, in part, in the X-ray beam 1308a. Portions of the resulting X-ray beam that do not pass through the treatment volume 1310 are omitted for clarity. Furthermore, at a second time, the electron beam 1304b may result, in part in the X-ray beam 1308b. Thus, for example, the treatment volume 1310 may be subject to a relatively higher radiation intensity, while the surrounding volumes may be subject to lower radiation intensities. Thus, for example, a treatment volume 1310, such as a tumor in a body or the like, may be targeted by a relatively stationary X-ray assembly.

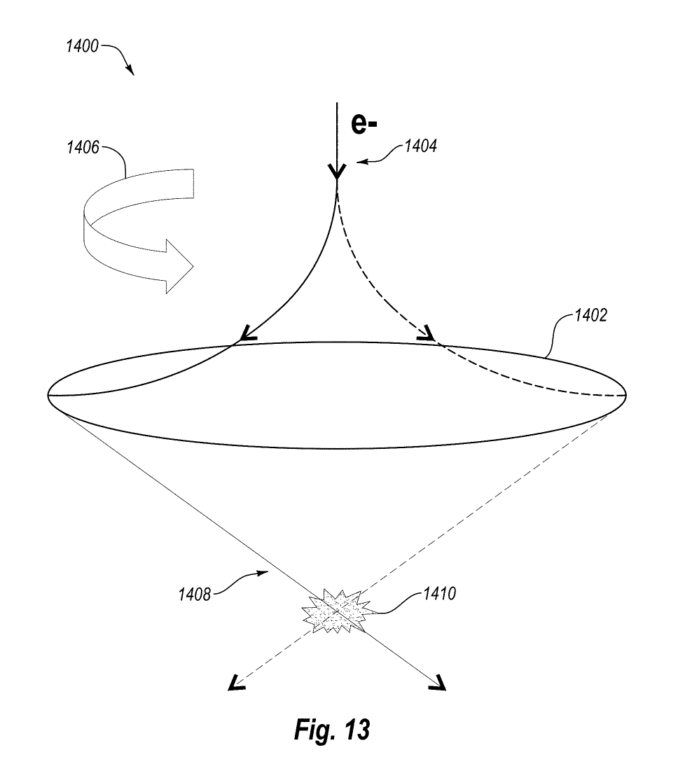

FIG. 13 is a diagram of an example X-ray system 1400. The X-ray system 1400 may be used to create a treatment volume 1410 in a manner analogous to the X-ray system 1300 of FIG. 12. Optionally, the X-ray system 1400 may be used with a collimator, such as a collimator as described with reference to any of FIGS. 8-12.

The X-ray system 1400 may include an electron beam 1404, an X-ray beam 1408, and a treatment volume 1410 generally corresponding to the electron beam 1304, the X-ray beam 1308, and the treatment volume 1310 of FIG. 12. The electron beam 1404 may be steering a relatively circular motion, represented by arrow 1406.

The X-ray system 1400 includes a target area 1402 of an anode of an X-ray assembly. Portions of the X-ray assembly other than the target area 1402 are omitted for clarity. The target area 1402 may have a ring shape. For example, the target area 1402 may have a ring shape analogous to the target area 616 of FIG. 5. Alternatively, the target area 1402 may have a different shape.

By way of example, the target area 1402 may have a target area shape corresponding to the target area 1106 of FIG. 10. Additionally, the X-ray system 1400 may include a collimator corresponding to the collimator 1102 of FIG. 10. Thus, for example, the electron beam 1404 may be rotated to scan a frustoconical target area 1402.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An X-ray assembly comprising:
a housing;
an anode located at least partially within the housing, the anode including a target area configured such that X-rays generated at the target area form an area-source X-ray beam; and
a cathode assembly located at least partially within the housing, the cathode assembly positioned to deliver electrons to the target area of the anode from a plurality of directions relative to the target area, the plurality of directions including at least two substantially opposite directions relative to the target area.

2. The X-ray assembly of claim 1, wherein the cathode assembly is positioned at an offset relative to an edge of the target area.

3. The X-ray assembly of claim 2, wherein the cathode assembly is positioned within an interior edge of the target area.

4. The X-ray assembly of claim 1, wherein the cathode assembly includes an elongate cathode shaped to correspond to a shape of the target area.

5. The X-ray assembly of claim 1, wherein the cathode assembly includes a plurality of cathodes.

6. The X-ray assembly of claim 1, wherein the cathode assembly includes a focusing electrode.

7. The X-ray assembly of claim 1, wherein the cathode assembly is configured to vary an intensity of an electron beam across the target area such that an intensity of the area-source X-ray beam is varied.

8. The X-ray assembly of claim 1, wherein the target area includes a surface area greater than 10 square millimeters.

9. The X-ray assembly of claim 1, wherein the target area is planar and includes an oval shape, a polygon shape, or a ring shape.

10. The X-ray assembly of claim 1, wherein the target area is non-planar and includes a cylindrical surface shape, a ring shape, a concave ring shape, or a frustoconical surface shape.

11. The X-ray assembly of claim 10, wherein the target area includes the frustoconical surface shape, including a smaller end and a larger end, and wherein the smaller end is positioned closer to an X-ray window of the housing than the larger end.

12. A system comprising:
an X-ray assembly configured to generate a circular area-source X-ray beam; and
a focusing collimator including a focusing passage defining a portion of the focusing collimator through which X-rays may pass substantially unattenuated, the focusing passage having a shape corresponding to that of a portion of a surface of an inverted cone.

13. The system of claim 12, wherein the X-ray assembly includes a layer of radioactive isotope positioned to generate the circular area-source X-ray beam.

14. The system of claim 12, wherein the focusing collimator includes a plurality of focusing passages including the focusing passage, each of the plurality of focusing passages having a shape corresponding to a portion of a surface of an inverted cone.

15. The system of claim 14, wherein the plurality of focusing passages are divergent.

16. The system of claim 14, wherein the plurality of focusing passages are convergent.

17. The system of claim 14, wherein the plurality of focusing passages are parallel.

18. The system of claim 12, wherein the X-ray assembly includes a plurality of point-source target areas positioned such that the plurality of point-source target areas are configured to collectively generate the circular area-source X-ray beam.

19. The system of claim 12, wherein the X-ray assembly includes an anode including a plurality of non-planar ring-shaped target areas.

20. The system of claim 12, wherein the X-ray assembly includes an anode including a concave-ring-shaped target area.

21. The system of claim 12, wherein the X-ray assembly includes an anode including a frustoconical-shaped target area.

22. The system of claim 21, wherein the X-ray assembly is configured to generate a rotating electron beam to scan the frustoconical-shaped target area.

23. The system of claim 12, wherein the focusing passage is configured to generate a treatment volume having a shape corresponding to a tumor of a patient.

24. The system of claim 23, wherein the focusing collimator includes a radial grid.

25. The system of claim 23, wherein the focusing collimator is manufactured using a three-dimensional printing technique.

26. A method of providing radiation treatment, the method comprising:
employing the system of claim 12 to introduce X-rays to a body from a plurality of different positions relative to the body.

27. An X-ray assembly comprising:
a cathode configured to generate an electron beam; and
an anode including a target,
wherein the X-ray assembly is configured to generate a magnetic or electric field positioned to bend the electron beam of the anode from an axis to incident on the target of the anode, the X-ray assembly being further configured to rotate the magnetic or electric field about the axis such that a location at which the electron beam incidents on the target of the anode is correspondingly rotated.

28. The X-ray assembly of claim 27 wherein the target of the anode comprises a transmission target.

29. The X-ray assembly of claim 27 wherein the target of the anode comprises a reflection target.
